(19) United States
(12) Patent Application Publication
(10) Pub. No.: US 2020/0196937 A1
(43) Pub. Date: Jun. 25, 2020

(54) Smart Cosmetic Applicator

(71) Applicant: Ramesh Gopalan, (US)

(72) Inventor: Ramesh Gopalan, Fremont, CA (US)

(73) Assignee: Ramesh Gopalan, Fremont, CA (US)

(21) Appl. No.: 16/720,098

(22) Filed: Dec. 19, 2019

Related U.S. Application Data
(60) Provisional application No. 62/782,300, filed on Dec. 19, 2018.

(51) Int. Cl. A61B 5/00; A45D 34/04; A45D 19/02; A45D 19/00; A45D 24/22

(52) U.S. Cl. CPC A61B 5/448; A45D 34/042; A45D 19/02; A45D 2044/007; A45D 24/22; A45D 2200/10; A45D 19/0008

(57) ABSTRACT

A smart applicator for liquid cosmetic to hair, skin or nails is described, comprising a position indicator to measure the relative change of position of the applicator across a nearly flat surface; a sensor array to measure the prior color or cosmetic condition of hair strands or skin condition; an optional comb array to direct hair strands from their roots into a regular linear array; a microcontroller which outputs electrical signals to operate an array of micro-mechanical or inkjet nozzles aligned with the comb array so as to dispense dye or cosmetic droplets to the hair strands or skin surface according to input received from said sensors.

### CROSS-REFERENCE TO RELATED APPLICATIONS

This application claims the benefit of provisional patent application Ser. No. 62/782,300 filed 2018 Dec. 19 by the present inventor.

Field of the invention: This invention pertains to the application of dyes, coloring or other cosmetic treatments to generate a controlled pattern on hair, skin or other keratinous surfaces.

### BACKGROUND OF THE INVENTION

Despite unprecedented advances in nearly all aspects of technology, methods and means of applying dye, coloring or cosmetics to human hair or skin have remained remarkably unchanged in recent decades. Commonly available hair care products generally include a tube of semi-solid dye and a brush or comb-like applicator through which the dye is squirted as pressure is applied to the tube. Another common method of applying hair dye or coloring products is soaking the longer strands of hair in larger volumes of viscous dye while sandwiched between aluminum foil in a technique known as balayage.

Hair coloring products can include harsh chemicals like ammonia, amines or hydrogen peroxide intended to lift up the scaly cuticle layer to expose hair tissue or cortex underneath so that dye pigments are effectively absorbed. Since contact with the underlying scalp or skin is to be avoided, coloring treatment is not able to reach the roots of the hair follicles in the scalp and so often leaving an unattractive residual contrast between the roots and the rest of the dyed hair strands. Further, gray hairs typically are sparsely spread amidst otherwise darker hair with original natural color. Since hair can grow typically a 0.5 inch-1 cm per month, this means that frequent treatments are needed to avoid undesirable contrast between individual gray strands or untreated roots and the treated areas of hair. Also in most cases, to address the individual strands of gray amidst darker surrounding hair requires covering the entire area with color, using far more dye than is needed, and requiring further effort to wash off excess afterward, costing additional time and money. Despite all the effort, the end result is typically unsatisfactory and means that images of not only regular folks but celebrities or politicians can still appear on television or social media with every detail of their imperfectly and incompletely treated hair revealed in high definition.

### DESCRIPTION OF RELATED AND PRIOR ART

Prior Art disclosures in this field include: US20100139682 and, ‘Devices and methods for modifying keratinous surfaces’ US20100224205 sensing the existing hair color and then selectively applying dye or coloring in response to the existing variation. Because position encoding or position registration are not included in these previous disclosures they do not address the challenge of controllably delivering the dye ink in proportion to the speed of traversal of the applicator across the uneven length of the hair strands or uneven surface of the scalp. If the volume of dye or cosmetic delivered per unit time is not adjustable according to the change of position per unit time then slowing down of the motion of dye applicator will lead to more dye than is necessary to treat the hair that has been traversed through, and speeding up would result in sparser application of dye. So the prior art does not allow the possibility of a manual application being able to render a controlled or pre-determined pattern by adjusting dose and timing of coloring on to an uneven or fibrous surface such as hair or skin. More recent disclosures include 14,736,551—Rabe et al and 14/736,584, granted as U.S. Pat. No. 9,522,101 and U.S. Pat. No. 10,188,192 in 2016 and 2019 respectively and assigned to Procter & Gamble co. on ‘Cartridges for the deposition of treatment compositions on keratinous surfaces’ describe delivery of cosmetics from a reservoir through nozzles controlled by a microcontroller or CPU after sensing of the skin at down to 10 um² level; again here there is no position encoder or position registration so does not allow the rendering of a pre-determined pattern onto the skin. Further, none of the published prior art describes schemes to isolate and treat single strands of hair which, like gray hairs, may be in contrast to a different background color.

The hair care products market in the United States is estimated at over $13 billion in 2018, with sales of home-use dyes/coloring products alone accounting for over $2 billion per year. (for example, ‘In Search of the Perfect Hair Dye’—NYTimes March 2018). The dominant trend on hair coloring products is towards using more natural or nature-derived ingredients to coat, not lift up, the hair cuticle layer to achieve the desired end treatment result.

### SUMMARY OF THE INVENTION

The current invention makes the application of cosmetic treatments to hair, skin as easy as the use of a (computer) mouse. By lifting and drawing hair from the roots into a comb array—like structure comprising teeth the present invention—a smart cosmetic applicator—is able to present each strand for treatment against an array of sub-millimeter sized nozzles which dispense liquid coloring or cosmetic through micro-mechanical means on to the individual strands, adjusting the timing, volume or dose of dye as needed while dispensing to needed target areas only. This avoids the waste of hair coloring and messiness with having to soak all the hair in color as is the case in current art. This invention includes an X-Y position indicator as a means of encoding and measuring rate of change of position of the applicator along the hair strands. This ability to register and measure rate of position or speed of traversal along the hair strands allows calibration and real-time adjustment of coloring dosage to be delivered to the nozzles: slow or fast delivery rate for slow or fast speed of traversal, respectively. Further, sensors may be included in the invention to measure the preceding variation in color or contrast on each individual strand at the leading edge of the applicator so as to adjust the subsequent delivery of coloring or cosmetic as necessary. The data stream from the these initial color state sensors and the position encoders is processed by a microcontroller or central processing unit CPU in order to send the appropriate output controlling electrical signals to the individual nozzles delivering liquid cosmetic. The delivery of artificial coloring or cosmetic to individual hair strands is thus controlled not only by the difference between the pre-existing and the pre-determined target color of the hair but also by the speed of traversal of the applicator across the scalp along the length of the hair strands.

Since the comb like structure comprises teeth that can be similar to that found in common hair or beard trimmers it can reach down to the roots in the scalp to lift the hair slightly to color it down to these roots. The use of sub-millimeter sized nozzles allows the dispensing of cosmetic down to the hair follicles, while avoiding any exposure to the human scalp.

This avoids the disadvantages of the current art of wasting dye on unneeded areas of the hair which require no treatment while providing the advantage of controlled and targeted delivery of cosmetic along individual hair strands to their roots, with dose calibrated and timed by speed of traversal of the applicator, to render a pre-determined pattern of coloring or cosmetic on to hair. With the comb excluded an alternate embodiment of the invention may be used to produce a pre-determined pattern on skin, such as to render a tattoo or body art. An alternate embodiment of the invention may also be used to produce a finely detailed pattern on other keratinous surfaces such as finger or toe nails.

### DESCRIPTION OF THE DRAWINGS

Figures provide explanatory details referenced in the following detailed description. Embodiments depicted in the drawings are illustrative but do not limit the scope of the invention as will be evident to those familiar with the art. Reference numbers are provided to indicate correspondence between reference elements.

FIG. 1—shows a cross-section of an embodiment of the smart cosmetic applicator including the following features;
100—the outer housing of the applicator
101—comb teeth in an array to lift from scalp and direct hair strands to be treated by the nozzles in an array 102 with which the comb teeth array 101 is aligned
103—electronic circuitry providing output electrical signals to control operation of the nozzle array
104—a microcontroller or central processing unit CPU providing output electrical signals to control the nozzle electronic circuitry 103 while receiving input signals from the position indicator sensor 105 and initial color state sensor 106.
107—a reservoir for liquid dye or cosmetic in fluid connection with the nozzle array 102.
108—a rechargeable battery or wired source of electric power supplying the microcontroller 104, the sensors 105 and 106, and the nozzle electronic circuitry 103 as needed.
109—Hair strands drawn from human scalp or skin surface 110 which are subsequently treated as they traverse through the comb teeth array and past the nozzle array.
111—showing the direction of traversal of the applicator through the hair strands

FIG. 2—a front view of the comb teeth array showing a single tooth 201 in comb array 101 with hair strands 109 being treated as they are drawn past nozzle array 102 in FIG. 1. Untreated individual hairs 209 are shown in the plurality of hair strands 109. Individual hair strands 211 are shown after treatment. Individual nozzles 202 are shown in the nozzle array 102. The position indicator sensor 105 and initial color state sensor 106 are shown. The direction of traversal of the applicator is denoted as 111.

FIG. 3—depicts a flow chart describing operation of an embodiment of the invention to apply cosmetic to hair strands being drawn from a skin surface through a comb array to be aligned with a nozzle array dispensing fluid cosmetic accordingly to prescribing electronic signals from a microcontroller which takes input signals from a position indicator sensor and or an initial state sensor.

FIG. 4—depicts a flow chart describing operation of an embodiment of the invention which provide cosmetic treatment to keratinous surfaces such as human skin or nails while not requiring a comb array.

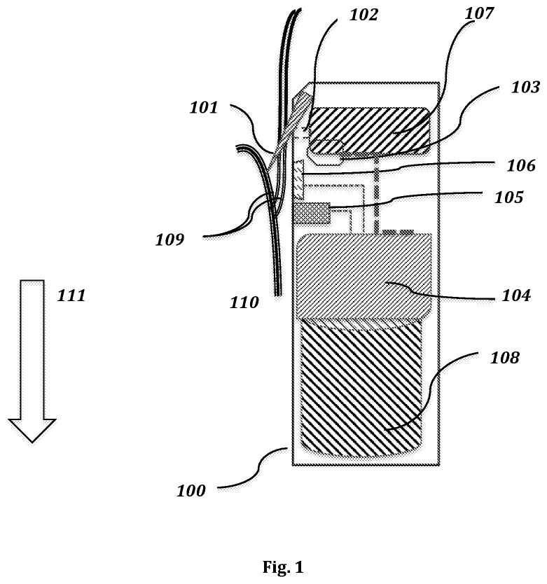

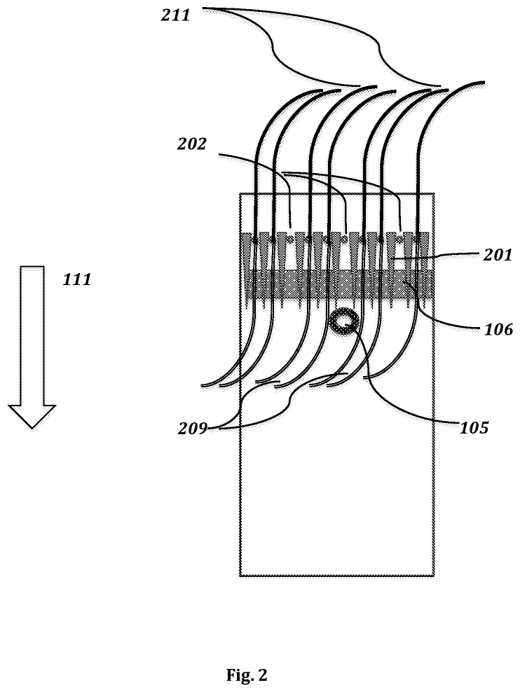

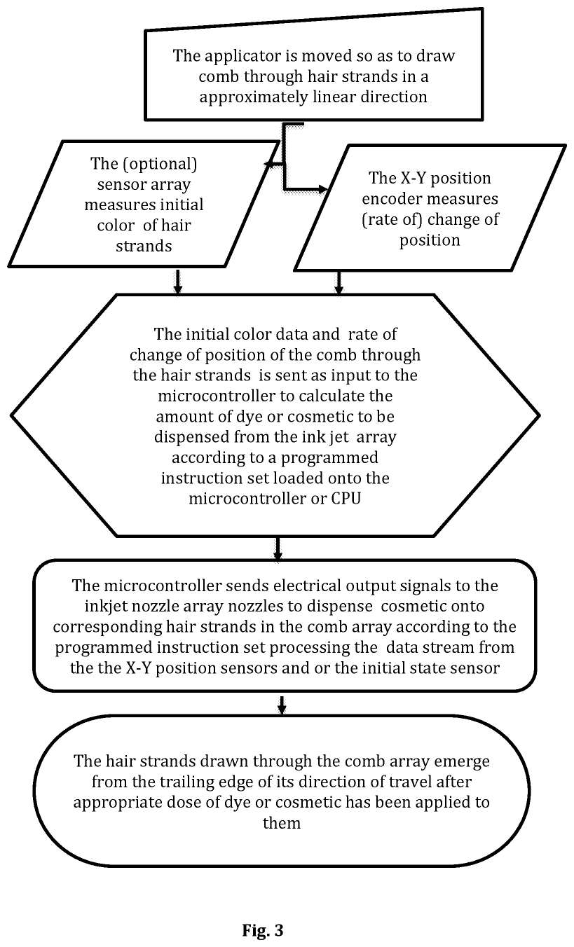

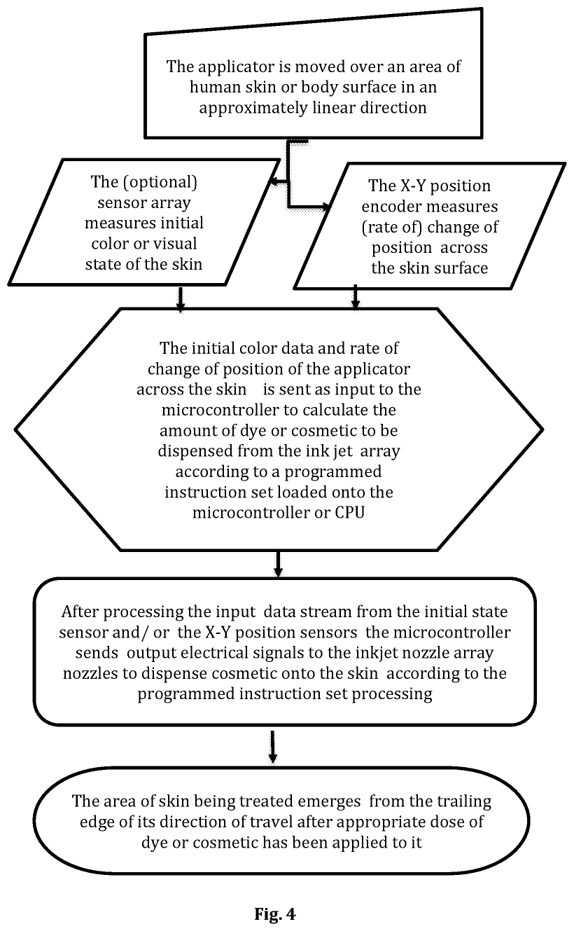

### DETAILED DESCRIPTION OF THE INVENTION

The typical human hair is a partially porous fiber made up of strands of the protein keratin and usually less than 100 microns=0.1 millimeter or four thousandths of an inch thick . Since there are typically around 600 hair follicles per square inch of human scalp or about 100 follicles per square centimeter we find that there are typically about 10 hair strands per linear centimeter (or 25 per linear inch) emerging from the typical human scalp. This is, of course, in agreement with common experience: a typical comb, for example, will have teeth spaced according to the typical hair spacing of 10 hairs/cm or 1 mm apart. The gap between adjacent teeth typically narrows or tapers from ˜1-2 mm at the widest end to about 0.1 mm—about the thickness of a hair strand—at its narrowest.

For some perspective on the dimensions involved, our current technological age of microelectronics has enabled billions of smartphones, each with about a billion transistors on silicon chips within them, each such transistor less than 1/1000th of a human hair in width. The 0.1 mm thickness and 1 mm spacing between hair strands is also very wide when compared with the capability of common inkjet or bubblejet printers which print can well over 1000 dots per linear inch (dpi), or one dot per 25 microns=0.025 mm, on to fibrous surfaces such as paper. The cellulose fibers that make up paper are also 10-100 microns thick and the printing process involves absorption of dispensed ink by these fibers. Coincidentally, the average human skin cell is about 30 microns in diameter, with about 1000 fitting in a linear inch.

The technology enabled by a common printer is delivering precise volumes of liquid ink or dye at precise times on to a paper moving past at a constant velocity, resulting in regularly placed ink spots, or dots. The registration of the position of these dots relative to each other enables the rendering of images or text in a precise and repeatable fashion. Inkjet or bubblejet printing has been in common use for over two decades. Inkjet printers for instance use a compact cartridge to dispense ink droplets into ‘dots’ on a page at densities of 100-1200 dots per inch (dpi). This means that the dots can be spaced about 0.02-0.25 mm apart, so that to dye human hair drawn through a comb into a linear array spaced about 1 mm apart the nozzles can be placed even wider than the lowest resolution printers currently available.

Therefore, to enable a smart cosmetic applicator that prints a color pattern onto hair just like a inkjet printer does on paper we would need the following features:
1. A comb like structure with teeth that lift the hair strands from their roots in the scalp while separating them into a regular linear array.
2. An array of sensors aligned with the comb array to sense or measure the existing or prior cosmetic condition or initial color state of the individual hair strands.
3. A position encoder or indicator that registers position and measures rate of change of position of the applicator on the (nearly) flat surface of the human scalp, or, alternatively, as a linear motion sensor, measures the rate of travel of the hair strands through and past the comb array; examples of such position encoders or motion sensors being as implemented in a common computer optical or laser mouse.
4. An array of inkjet nozzles or other micro-mechanical means of dispensing color or liquid cosmetic through said nozzles in fluid connection with a reservoir of said color or liquid cosmetic; said nozzle array also being aligned with the comb array.
5. A microcontroller or microprocessor unit providing signals to the inkjet nozzles to dispense liquid dye droplets according to feedback from the position encoder ; feedback from the sensor array and/or according to pre-determined recipe or pattern.

This technology would provide the following advantages over the prior art:
1. Use micro-mechanical means , such as inkjet nozzles, of delivering highly targeted, precise doses of cosmetic precisely to the locations where hair treatment is needed.
2. Use sensors that detect color or contrast, much like common barcode scanners, to detect and target for cosmetic application only those hairs that need it., while leaving others untouched. This may be used to also match coloring on treated hairs to those next to it, minimizing contrast and maximizing a natural look for the treated hair. This enables targeting individual hairs, providing color or cosmetic to these according to their need. Individual gray hairs may be treated with color, for instance, while avoiding the need to apply broad swathes of color or cosmetic treatment where it is not needed.
3. Access the hairs at their roots, holding and lifting each strand like a common hair trimmer, while avoiding cosmetic being applied to the scalp, skin or other sensitive areas.
4. Use position encoders, indicators or motion sensors to determine the speed of traversal of the applicator nozzles across the scalp or length of the hair strands so that the delivery of color or cosmetic is compensated or adjusted accordingly—with the flux and volume of liquid dye or color dispensed in unit time being proportional to the linear speed of the applicator traversal or distance covered in unit time.

The comb structure that separates the hairs approximately 1 mm apart can be as simple as those seen in common hair trimmers but with the additional feature such that the comb teeth will be at a small, shallow angle to the scalp, and also be tilted slightly from the direction of travel so that linear motion of the applicator achieves the outcome of drawing the hair strands from the roots into the dye stream of the nozzles; the tilt/slant of the comb teeth ensuring that the hairs are lifted up slightly into the proximity of the nozzles that they may receive the dispensed dye droplets reliably.

The sensor array could be a scanner for contrast, such a common barcode scanner, which identifies the pre-existing coloring of the hair for subsequent treatment as needed and also providing to the microprocessor the position of the hairs in the comb array that need dye dispensed on them.

The inkjet nozzle sizes can be chosen to eject dye droplets that are bigger than the width of the widest possible hair (>100 um) so as to wet and coat the entire surface of the hair strand, for effective dye or cosmetic coverage. The dye must wet and diffuse through the ˜5 micron thick outer cuticle to dye the hair cortex which holds moisture and melanin color pigment, underneath. It is important to deliver just the right amount of dye per unit length of hair: neither too much, which would drip off the hair, nor too little, to result in inadequate coloring of the strands. Therefore, it is important to adjust the delivery of dye droplets from the nozzle according to the speed of traversal or rate of travel of the hair through the comb array.

The sensor which detects the prior or initial color of the hair strands may be located at the leading edge of the applicator in the direction of travel so as to feed information to the microcontroller about which hair strands in the linear comb array need treatment from the nozzles that may be located in a section trailing the color or contrast sensor, in the typical direction of motion of the applicator. The position indicator, encoder or linear motion sensor may also be located near the color or contrast sensor at the leading edge of the applicator near where the applicator first meets the hair to be treated. The comb array or structure may also be located so as to have the narrow tip of its teeth towards the leading edge of the applicator so as to lift up the hair strands and draw them into an array for sensing in a regular pattern by the color and linear motion sensors and subsequently towards the individual nozzles in the nozzle array.

The principle behind inkjet printing is well known. A sub-millimeter sized tube draws in liquid ink or dye through capillary action. A pulse of current through a thin-film resistive heating element at one end of the tube causes a vapor bubble to form and this volume expansion causes a droplet of dye or ink to be ejected from the nozzle at the other end of the tube. For the inkjet principle to work the dye needs to include at least one volatile component, a criterion that is easily met by most cosmetics or liquid dyes on the market today. The other micro-mechanical method —the bubble jet—achieves dispensing ink or dye through small nozzles by using a piezo-electric actuator whose vibration produces a liquid stream, which break up into uniformly sized droplets (according to theory first explained by Lord Rayleigh in 1878).

Just with the case of inks used to print on paper, minor modifications to the dye composition, including inert and non-toxic components, may be necessary for optimal dispensing of dye droplets onto hair. As described in Inkjet applications by Matt Gilliland, for example, common inkjet cartridge nozzles are activated by a ˜20V pulse, lasting about 5 microseconds (us) (with about 800 us delay on each nozzle, to allow the nozzles to recharge or refill with dye or liquid ink. There is also typically a 0.5 us delay required before the electronics can trigger or fire another nozzle. The microcontroller may be programmed to trigger such pulses depending on feedback from the various sensors of the applicator—the initial color state sensor and the position encoder. For this purposes of this invention, both the inkjet or bubblejet methods may be used to dispense dye through nozzles according to signals from the microcontroller.

For the position encoder or indicator, we take instruction from the common optical or laser mouse used with computers. Electromechanical or laser optical mice (plural for mouse) have been known since Doug Engelbart's X-Y position indicator for a display system' filed in 1967 (U.S. Pat. No. 3,541,541 granted in 1970) to map the motion on a nearly flat X-Y surface to translate it into, for example, the motion of a cursor on a display for a computer, or in the present case, used to calibrate or adjust the dispensing of dye or liquid cosmetic according to the speed of traversal of the applicator nozzles across the hair strands. (Famously, the mouse was demonstrated at Xerox PARC to Steve Jobs, co-founder of Apple with Steve ‘Woz’niak, who, like Engelbart, had graduated from the University of California, Berkeley)

A typical optical mouse will use the light from a light-emitting diode LED and track relative motion on a surface or substrate by measuring the shift of the reflected light pattern from said surface. A similar principle of specular reflection is used with higher positional accuracy with so-called laser mouse which is capable of position resolution of up to 6000 dot (or pixels) per inch, higher than the more common optical mouse which is generally capable of about 3000 dots per inch (dpi)—this spatial resolution of about 10 microns is adequate to resolve not only individual hair strands but typical human skin cells as well.

Since the typical hair is 4 thousandths of an inch thick, or about 12 dots wide (for an optical mouse; 25 dots wide for a laser mouse) this enables tracking of the traversal rate (speed) and distance traversed and traversal rate (speed) of the relative motion of each hair strand as it passes the comb array.

For the contrast or color sensor commonly available CCD sensors may be used which, coupled with the right color filters may be used to detect not only contrast on a gray-scale but also Red-Green-Blue (RGB) color levels. Such a implementation in common barcode scanners which read an array of dark-light bars used for identification in common shipping or retail applications.

Since both the color sensor and position sensor use common elements such as a source of illumination(an LED or laser) and a method of detecting reflected light off the surface (e.g. a CCD array)—they may be combined into one unit to optimize the design of the applicator. The invention may be modified to include additional functionalities such as LEDs or lasers which provide localized heating of hair strands. Also, localized streams of ions or electrons may be generated to apply electric charge to the hair strands for the purpose of separating the hair strands, as is common in some implementations of inkjet printing.

The length and shape of the comb teeth may be designed to lift hair strands to draw them to the inkjet nozzles to be treated with cosmetic. These may be made retractable so that their length may be adjusted so as to enable the applicator reaching the hair roots, much as a common beard trimmer or electric shaver access hair at their follicles on human scalp or face.

It is noted that the alternate embodiment of the invention may include only the position indicator sensor with the initial color state sensor being optional.

Human skin, like hair, is also made up of dead keratinous cells on its outer surface exposed to the environment. It will be clear to those familiar with the art that, by excluding the comb array unnecessary in this case, the present invention may be used to render a pre-determined pattern of cosmetic, medication or coloring to the uneven, yet locally flat, surface of human skin to produce a tattoo or similar body art. In this case, the spacing of the inkjet or micromechanical nozzles dispensing cosmetic or coloring will tend to be closer depending on the level of detail needed may be comparable to the typical human skin cell at about 30 microns in size, or be spaced at inkjet printer resolution of up to 1000 dots per inch (dpi).

An alternate embodiment of this invention may also be used to produce a finely detailed pattern or image on other keratinous surfaces including human finger or toe nails.

What is claimed is:

1. An applicator for applying liquid cosmetics or coloring to hair comprising:
a. A comb array comprising a plurality of comb teeth as means to lift hair strands at their roots whereby individual hair strands are separated into a regular linear array
b. a plurality of nozzles aligned in an array and disposed to dispense color or liquid cosmetics onto hair strands in said comb array
c. each such nozzle coupled to an inkjet or other micro-mechanical means of dispensing liquid cosmetic or coloring
d. a reservoir of coloring or cosmetics in fluid connection with said nozzle array
e. a X-Y position indicator as means to measure change of position of applicator upon a mostly flat surface such as a human scalp, whereby rate and direction of travel of each hair strand through the comb array is determined.
f. a microcontroller to output electrical signals to inkjet or micro-electromechanical nozzles to dispense color or liquid cosmetics as function of input signals from said position indicator and or according to pre-determined pattern.

2. The smart cosmetic applicator of claim 1 comprising a plurality of sensors in an array as means to measure initial color or other state of hair strands as they are drawn into the said comb array and
a microcontroller to output electrical signals to inkjet or micro-mechanical nozzles to dispense color as function of input signals from said position indicator, said sensors of initial state, and or according to pre-determined pattern

3. An applicator for applying cosmetic to skin comprising
a. a plurality of nozzles aligned in an array and disposed to dispense liquid cosmetics onto the skin surface
b. each such nozzle coupled to an inkjet or other micro-mechanical means of dispensing liquid cosmetic
c. a reservoir of cosmetics in fluid connection with said nozzle array
d. a X-Y position indicator as means to measure change of position of applicator upon a mostly flat surface, whereby rate and direction of travel of applicator across a human skin surface is determined
e. a microcontroller to output electrical signals to inkjet or micro-electromechanical nozzles to dispense color or liquid cosmetics as function of input signals from said position indicator and or according to pre-determined pattern.

4. The smart cosmetic applicator of claim 3 comprising a plurality of sensors in an array as means to measure initial color or other state of skin surface and a microcontroller to output electrical signals to inkjet or micro-mechanical nozzles to dispense cosmetic to the skin surface as function of input signals from said position indicator, said sensors of initial state, and or according to pre-determined pattern.

5. The applicator of claim 3 being configured to apply a pattern to a keratinous surface such as finger or toe nails.

6. The applicator of claim 4 being configured to apply a pattern to a keratinous surface such as finger or toe nails.

7. The comb teeth of claim 1 being retractable.

8. The applicator of claim 3 being used to render a skin tattoo or body art.
